(19) United States
(12) Patent Application Publication
(10) Pub. No.: US 2024/0050452 A1
(43) Pub. Date: Feb. 15, 2024

(54) USE OF CANNABIDIOL IN THE TREATMENT OF SEIZURES ASSOCIATED WITH EPILEPSY SYNDROMES IN PATIENTS TAKING BRIVARACETAM

(71) Applicant: GW Research Limited, Cambridgeshire (GB)

(72) Inventors: Kevin James CRAIG, Cambridgeshire (GB); Daniel Adam CHECKETTS, Cambridgeshire (GB)

(21) Appl. No.: 18/256,307

(22) PCT Filed: Dec. 8, 2021
(86) PCT No.: PCT/GB2021/053203

§ 371 Date: Jun. 7, 2023

(30) Foreign Application Priority Data
Dec. 8, 2020 (GB) 2019301.7

(51) Int. Cl. A61K 31/00; A61K 31/4015; A61K 36/185; A61K 45/06; A61P 25/08

(52) U.S. Cl. CPC A61K 31/658; A61K 31/4015; A61K 36/185; A61K 45/06; A61P 25/08

(57) ABSTRACT

The present invention relates to the use of cannabidiol (CBD) and an anti-epileptic drug (AED), brivaracetam (BRV), for the treatment of seizures associated with epilepsy syndromes. In particular the types of seizures treated include atonic, tonic, tonic-clonic, and focal seizures with secondary generalisation. Preferably the dose of CBD is between 5 mg/kg/day to 50 mg/kg/day.

### FIELD OF THE INVENTION

The present invention relates to the use of cannabidiol (CBD) and an anti-epileptic drug (AED), brivaracetam (BRV), for the treatment of seizures associated with epilepsy syndromes. In particular the types of seizures treated include atonic, tonic, tonic-clonic, and focal seizures with secondary generalisation. Preferably the dose of CBD is between 5 mg/kg/day to 50 mg/kg/day.

In a further embodiment the CBD used is in the form of a highly purified extract of cannabis such that the CBD is present at greater than 95% of the total extract (w/w) and the cannabinoid tetrahydrocannabinol (THC) has been substantially removed, to a level of not more than 0.15% (w/w).

Preferably the CBD used is in the form of a botanically derived purified CBD which comprises greater than or equal to 98% (w/w) CBD and less than or equal to 2% (w/w) of other cannabinoids. More preferably the other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used.

Where the CBD is given concomitantly with one or more other anti-epileptic drugs (AED), the CBD may be formulated for administration separately, sequentially or simultaneously with one or more AED or the combination may be provided in a single dosage form.

### BACKGROUND TO THE INVENTION

Epilepsy occurs in approximately 1% of the population worldwide, (Thurman et al., 2011) of which 70% are able to adequately control their symptoms with the available existing anti-epileptic drugs (AED). However, 30% of this patient group, (Eadie et al., 2012), are unable to obtain seizure freedom from the AED that are available and as such are termed as suffering from intractable or “treatment-resistant epilepsy” (TRE).

Intractable or treatment-resistant epilepsy was defined in 2009 by the International League Against Epilepsy (ILAE) as “failure of adequate trials of two tolerated and appropriately chosen and used AED schedules (whether as monotherapies or in combination) to achieve sustained seizure freedom” (Kwan et al., 2009).

Individuals who develop epilepsy during the first few years of life are often difficult to treat and as such are often termed treatment resistant. Children who undergo frequent seizures in childhood are often left with neurological damage which can cause cognitive, behavioral and motor delays.

Childhood epilepsy is a relatively common neurological disorder in children and young adults with a prevalence of approximately 700 per 100,000. This is twice the number of epileptic adults per population.

When a child or young adult presents with a seizure, investigations are normally undertaken in order to investigate the cause. Childhood epilepsy can be caused by many different syndromes and genetic mutations and as such diagnosis for these children may take some time.

The main symptom of epilepsy is repeated seizures. In order to determine the type of epilepsy or the epileptic syndrome that a patient is suffering from an investigation into the type of seizures that the patient is experiencing is undertaken. Clinical observations and electroencephalography (EEG) tests are conducted and the type(s) of seizures are classified according to the ILEA classification.

Generalized seizures, where the seizure arises within and rapidly engages bilaterally distributed networks, can be split into six subtypes: tonic-clonic (grand mal) seizures; absence (petit mal) seizures; clonic seizures; tonic seizures; atonic seizures and myoclonic seizures.

Focal (partial) seizures where the seizure originates within networks limited to only one hemisphere, are also split into sub-categories. Here the seizure is characterized according to one or more features of the seizure, including aura, motor, autonomic and awareness/responsiveness. Where a seizure begins as a localized seizure and rapidly evolves to be distributed within bilateral networks this seizure is known as a bilateral convulsive seizure, which is the proposed terminology to replace secondary generalized seizures (generalized seizures that have evolved from focal seizures and are no longer remain localized).

Focal seizures where the subject's awareness/responsiveness is altered are referred to as focal seizures with impairment and focal seizures where the awareness or responsiveness of the subject is not impaired are referred to as focal seizures without impairment.

Cannabidiol (CBD), a non-psychoactive derivative from the cannabis plant, has demonstrated anti-convulsant properties in several anecdotal reports, pre-clinical and clinical studies both in animal models and humans. Three randomized control trials showed efficacy of the purified pharmaceutical formulation of CBD in patients with Dravet and Lennox-Gastaut syndrome.

Based on these three trials, a botanically derived purified CBD preparation was approved by FDA in June 2018 for the treatment of seizures associated with Dravet and Lennox-Gastaut syndromes.

Brivaracetam (BRV) is an adjunctive therapy for the treatment of focal seizures with or without secondary generalisation.^{1,2} It is approved for use alone or with other seizure medicines in adults and children 4 years of age and older with focal (partial) seizures. Brivaracetam belongs to a class of medications called anticonvulsants, which work by decreasing abnormal electrical activity in the brain.

A study by Klotz et al. in 2019 investigated the effect of CBD on BRV plasma levels³. Results showed that the combination of an increasing CBD dose with a constant BRV dose led to reductions in seizure frequencies, although the exact seizure types affected are not disclosed. The report neither discloses nor suggests the composition of the CBD preparation used.

WO 2016/160542 discloses compositions for treating seizure disorders using the combination of a non-barbiturate anti-epileptic drug (NAED), CBD, and a lipophilic fatty acid, with BRV being listed as a potential NAED in a list of 27 NAEDs. The document does not provide any data on the efficacy of such a combination.

WO 2019/180706 discloses cannabinoid combinations comprising CBD and cannabidivarin (CBDV), at a weight ratio of from about 10:1 to about 20:1, for use in treating epilepsy. BRV is mentioned as a potential adjunctive anti-epileptic drug. There is no data to show efficacy of a cannabinoid composition with BRV specifically. The main finding of the document was that a cannabis extract with CBD, CBDV and THC at a ratio of 30:2:1 (75, 5 and 2.5%) was most effective in a few seizure types.

WO 2020/225540 and GB 2539472 disclose the use of highly purified CBD in the treatment of seizures associated with epileptic syndromes including Tuberous Sclerosis Syndrome and Dravet Syndrome. Neither disclose any efficacy data of BRV in combination with CBD.

The applicant has found by way of an open label, expanded-access program that treatment with CBD combined with a reduced dose of brivaracetam resulted in a significant reduction in specific seizure types including atonic, tonic, tonic-clonic, and focal seizures with secondary generalisation in patients with epilepsy syndrome.

### BRIEF SUMMARY OF THE DISCLOSURE

In accordance with a first aspect of the present invention there is provided a cannabidiol (CBD) preparation for use in the treatment of seizures associated with epilepsy in patients who are concurrently taking brivaracetam wherein the dose of brivaracetam is lowered.

In a further embodiment the dose of the CBD preparation is lowered.

In a further embodiment the seizures associated with epilepsy are atonic, tonic, tonic-clonic seizures and focal seizures with secondary generalisation.

In a further embodiment, the CBD preparation comprises greater than 95% (w/w) CBD, not more than 0.15% (w/w) tetrahydrocannabinol (THC) and up to 1% cannabidivarin (CBDV).

Preferably the CBD preparation comprises greater than or equal to 98% (w/w) CBD and less than or equal to 2% (w/w) other cannabinoids, wherein the less than or equal to 2% (w/w) other cannabinoids comprise the cannabinoids tetrahydrocannabinol (THC); cannabidiol-C1 (CBD-C1); cannabidivarin (CBDV); and cannabidiol-C4 (CBD-C4), and wherein the THC is present as a mixture of trans-THC and cis-THC.

Preferably the CBD preparation is used in combination with one or more concomitant anti-epileptic drugs (AED).

Preferably the one or more AED is selected from the group consisting of: clobazam, lamotrigine, lacosamide, rufinamide, levetiracetam, diazepam, felbamate and clonazepam.

In one embodiment the CBD is present is isolated from cannabis plant material. Preferably at least a portion of at least one of the cannabinoids present in the CBD preparation is isolated from cannabis plant material.

In a further embodiment the CBD is present as a synthetic preparation. Preferably at least a portion of at least one of the cannabinoids present in the CBD preparation is prepared synthetically.

Preferably the dose of CBD is greater than 5 mg/kg/day. More preferably the dose of CBD is 20 mg/kg/day. More preferably the dose of CBD is 25 mg/kg/day. More preferably the dose of CBD is 50 mg/kg/day.

In accordance with a second aspect of the present invention there is provided a method of treating seizures associated with epilepsy in a patient in need thereof, comprising administering to the patient a therapeutically effective amount of cannabidiol with caution, wherein the patient is taking brivaracetam concurrently.

In a further embodiment the caution comprises lowering the dose of CBD.

In a further embodiment the caution comprises monitoring said patient for side effects and discontinuing CBD if said side effects are observed.

In a further embodiment the caution comprises advising said patient of side effects from said concurrent therapy.

### Definitions

Definitions of some of the terms used to describe the invention are detailed below:

Over 100 different cannabinoids have been identified, see for example, Handbook of Cannabis, Roger Pertwee, Chapter 1, pages 3 to 15. These cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to 95% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

“Treatment-resistant epilepsy” (TRE) or “intractable epilepsy” is defined as per the ILAE guidance of 2009 as epilepsy that is not adequately controlled by trials of one or more AED.

“Atonic seizures” occur when a person suddenly loses muscle tone so their head or body may go limp. They are also known as drop attacks. In some children, only their head drops suddenly. They can begin in one area or side of the brain (focal onset) or both sides of the brain (generalized onset).

“Tonic seizures” can be generalised onset, affecting both sides of the brain, or they can be focal onset, starting in just one side of the brain. If a tonic seizure starts in both sides of the brain, all muscles tighten and the subject's body goes stiff. If standing, they may fall to the floor, their neck may extend, eyes open wide and roll upwards, whilst their arms may raise upwards and legs stretch or contract. If a tonic seizure starts in one side of the brain muscles tighten in just one area of the body. Tonic seizures usually last less than one minute.

“Tonic-clonic seizures” consist of two phases: the tonic phase and the clonic phase. In the tonic phase the body becomes entire rigid, and in the clonic phase there is uncontrolled jerking. Tonic-clonic seizures may or may not be preceded by an aura, and are often followed by headache, confusion, and sleep. They may last mere seconds or continue for several minutes. These seizures are also known as a grand mal seizure.

“Focal Seizures” are defined as seizures which originate within networks limited to only one hemisphere. What happens during the seizure depends on where in the brain the seizure happens and what that part of the brain normally does.

“Focal seizure with secondary generalisation” start in a limited area on one side of the brain and spread to involve both sides. This is different from a generalized onset seizure, which starts on both sides of the brain.

“Focal seizure with impairment” usually start in a small area of the temporal lobe or frontal lobe of the brain and involve other areas of the brain within the same hemisphere that affect alertness and awareness. Most subjects experience automatisms during a focal seizure with impaired consciousness.

### DETAILED DESCRIPTION

### Preparation of Highly Purified CBD Extract

The following describes the production of the highly-purified (>95% w/w) cannabidiol extract which has a known and constant composition.

In summary the drug substance used is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 95% CBD. Although the CBD is highly purified because it is produced from a cannabis plant rather than synthetically there is a small number of other cannabinoids which are co-produced and co-extracted with the CBD. Details of these cannabinoids and the quantities in which they are present in the medication are as described in Table A below.

TABLE A
Composition of highly purified CBD extract

| Cannabinoid | Concentration |
|---|---|
| CBD | >95% w/w |
| CBDA | NMT 0.15% w/w |
| CBDV | NMT 1.0% w/w |
| Δ⁹ THC | NMT 0.15% w/w |
| CBD-C4 | NMT 0.5% w/w |

>—greater than
NMT—not more than

### Preparation of Botanically Derived Purified CBD

The following describes the production of the botanically derived purified CBD which comprises greater than or equal to 98% w/w CBD and less than or equal to other cannabinoids was used in the open label, expanded-access program described in Example 1 below.

In summary the drug substance used in the trials is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 95% CBD w/w, typically greater than 98% w/w.

The Cannabis sativa L. plants are grown, harvested, and processed to produce a botanical extract (intermediate) and then purified by crystallization to yield the CBD (botanically derived purified CBD).

The plant starting material is referred to as Botanical Raw Material (BRM); the botanical extract is the intermediate; and the active pharmaceutical ingredient (API) is CBD, the drug substance.

All parts of the process are controlled by specifications. The botanical raw material specification is described in Table B and the CBD API is described in Table C.

TABLE B
CBD botanical raw material specification

| Test | Method | Specification |
|---|---|---|
| Identification: | Visual | Complies |
| A | TLC | Corresponds to |
| B | HPLC/UV | standard (for CBD & CBDA) |
| C | | Positive for CBDA |
| Assay: | In-house | NLT 90% of assayed |
| CBDA + CBD | (HPLC/UV) | cannabinoids by peak area |
| Loss on Drying | Ph. Eur. | NMT 15% |
| Aflatoxin | UKAS method | NMT 4 ppb |
| Microbial: | Ph. Eur. | NMT10⁷ cfu/g |
| TVC | | NMT10⁵ cfu/g |
| Fungi | | NMT10² cfu/g |
| E. coli | | |
| Foreign Matter: | Ph. Eur. | NMT 2% |
| Residual Herbicides and | Ph. Eur. | Complies |
| Pesticides | | |

TABLE C
Specification of an exemplary botanically
derived purified CBD preparation

| Test | Test Method | Limits |
|---|---|---|
| Appearance | Visual | Off-white/pale yellow crystals |
| Identification A | HPLC-UV | Retention time of major |
| | | peak corresponds to |
| | | certified CBD Reference Standard |
| Identification B | GC-FID/MS | Retention time and mass |
| | | spectrum of major peak |
| | | corresponds to certified |
| | | CBD Reference Standard |
| Identification C | FT-IR | Conforms to reference |
| | | spectrum for certified |
| | | CBD Reference Standard |
| Identification D | Melting Point | 65-67° C. |
| Identification E | Specific | Conforms with certified |
| | Optical | CBD Reference |
| | Rotation | Standard; −110° to −140° |
| | | (in 95% ethanol) |
| Total Purity | Calculation | ≥98.0% |
| Chromatographic | HPLC-UV | ≥98.0% |
| Purity 1 | | |
| Chromatographic | GC-FID/MS | ≥98.0% |
| Purity 2 | | |
| CBDA | HPLC-UV | NMT 0.15% w/w |
| CBDV | | NMT 1.0% w/w |
| THC | | NMT 0.1% w/w |
| CBD-C4 | | NMT 0.5% w/w |
| Residual Solvents: | GC | NMT 0.5% w/w |
| Alkane | | NMT 0.5% w/w |
| Ethanol | | |
| Residual Water | Karl Fischer | NMT 1.0% w/w |

The purity of the botanically derived purified CBD preparation was greater than or equal to 98%. The botanically derived purified CBD includes THC and other cannabinoids, e.g., CBDA, CBDV, CBD-C1, and CBD-C4.

Distinct chemotypes of the Cannabis sativa L. plant have been produced to maximize the output of the specific chemical constituents, the cannabinoids. Certain chemovars produce predominantly CBD. Only the (−)-trans isomer of CBD is believed to occur naturally. During purification, the stereochemistry of CBD is not affected.

### Production of CBD Botanical Drug Substance

An overview of the steps to produce a botanical extract, the intermediate, are as follows:
a) Growing
b) Direct drying
c) Decarboxylation
d) Extraction—using liquid CO₂
e) Winterization using ethanol
f) Filtration
g) Evaporation

High CBD chemovars were grown, harvested, dried, baled and stored in a dry room until required. The botanical raw material (BRM) was finely chopped using an Apex mill fitted with a 1 mm screen. The milled BRM was stored in a freezer prior to extraction.

Decarboxylation of CBDA to CBD was carried out using heat. BRM was decarboxylated at 115° C. for 60 minutes.

Extraction was performed using liquid CO₂ to produce botanical drug substance (BDS), which was then crystalized to produce the test material. The crude CBD BDS was winterized to refine the extract under standard conditions (2 volumes of ethanol at −20° C. for approximately 50 hours). The precipitated waxes were removed by filtration and the solvent was removed to yield the BDS.

### Production of Botanically Derived Purified CBD Preparation

The manufacturing steps to produce the botanically derived purified CBD preparation from BDS were as follows:
a) Crystallization using C₅-C₁₂ straight chain or branched alkane
b) Filtration
c) Vacuum drying

The BDS produced using the methodology above was dispersed in C₅-C₁₂ straight chain or branched alkane. The mixture was manually agitated to break up any lumps and the sealed container then placed in a freezer for approximately 48 hours. The crystals were isolated via vacuum filtration, washed with aliquots of cold C₅-C₁₂ straight chain or branched alkane, and dried under a vacuum of <10 mb at a temperature of 60° C. until dry. The botanically derived purified CBD preparation was stored in a freezer at −20° C. in a pharmaceutical grade stainless steel container, with FDA food grade approved silicone seal and clamps.

### Physicochemical Properties of the Botanically Derived Purified CBD

The botanically derived purified CBD used in the clinical trial described in the invention comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w).

The botanically derived purified CBD used additionally comprises a mixture of both trans-THC and cis-THC. It was found that the ratio of the trans-THC to cis-THC is altered and can be controlled by the processing and purification process, ranging from 3.3:1 (trans-THC:cis-THC) in its unrefined decarboxylated state to 0.8:1 (trans-THC:cis-THC) when highly purified.

Furthermore, the cis-THC found in botanically derived purified CBD is present as a mixture of both the (+)-cis-THC and the (−)-cis-THC isoforms.

Clearly a CBD preparation could be produced synthetically by producing a composition with duplicate components.

Example 1 below describes the use of a botanically derived purified CBD in an open label, expanded-access program to investigate the clinical efficacy and safety of purified pharmaceutical cannabidiol formulation (CBD) in the treatment of patients with epilepsy who are concurrently taking brivaracetam.

### Example 1: Clinical Efficacy and Safety of Purified Pharmaceutical Cannabidiol (CBD) and Brivaracetam in the Treatment of Patients with Epilepsy

### Study Design

Subjects were required to be on one or more AEDs at stable doses for a minimum of two weeks prior to baseline and to have stable vagus nerve stimulation (VNS) settings and ketogenic diet ratios for a minimum of four weeks prior to baseline.

Patients were administered botanically derived purified CBD in a 100 mg/mL sesame oil-based solution which was titrated to an initial dose of between 5 and 25 milligrams per kilogram per day (mg/kg/day) in two divided doses. Doses were then increased weekly by 5 mg/kg/day to a goal of 25 mg/kg/day where required.

A maximum dose of 50 mg/kg/day could be utilised for patients who were tolerating the medication but had not achieved seizure control; these patients had further weekly titration by 5 mg/kg/day.

There were three patients in this study, and each received CBD and BRV for various durations of time. Modifications were made to concomitant AEDs as per clinical indication.

Seizure frequency, intensity, and duration were recorded by caregivers in a diary during a baseline period of at least 28 days. Changes in seizure frequency relative to baseline were calculated after at least 2 weeks and at defined timepoints of treatment.

### Statistical Methods:

The percent change in seizure frequency was calculated as follows:

$$\% change seizure frequency = \frac{\begin{matrix}( weekly seizure frequency time interval ) - \\ ( weekly seizure frequency Baseline )\end{matrix}}{( weekly seizure frequency Baseline )}$$

The percent change of seizure frequency may be calculated for any time interval where seizure number has been recorded. For the purpose of this example the percent change of seizure frequency for the specified time intervals was calculated as follows:

$$\% reduction seizure frequency = \frac{( weekly seizure frequency x ) - ( weekly seizure frequency y )}{( weekly seizure frequency y )}$$

### Results

### Patient Description

The three patients enrolled in the open label; expanded-access program had epilepsy. These patients experienced a range of different seizure types including atonic, tonic, tonic-clonic, focal seizures with secondary generalisation and focal seizures with impairment.

The age of patients ranged from 9-15 years, two were male and one was female as detailed in Table 1 below.

TABLE 1
Patient demographics, seizure type and concomitant medication

| Patient | Age | | | |
|---|---|---|---|---|
| Number | (years) | Sex | Seizure types | Concomitant AEDs |
| 1 | 15.36 | M | Focal with secondary | CLB, LMT, LAC |
| | | | generalisation | |
| 2 | 13.75 | M | Tonic | CLB, RFN |
| | | | Tonic-clonic | |
| 3 | 8.89 | F | Atonic | LEV, RFN, LMT, |
| | | | Tonic | DZP, FLB, CLZ |
| | | | Focal with impairment | |

CLB = clobazam,
LMT = lamotrigine,
LAC = lacosamide,
RFN = rufinamide,
LEV = levetiracetam,
DZP = diazepam,
FLB = felbamate,
CLZ = clonazepam

### Study Medication and Concomitant Medications

Patients on the study were titrated up to various doses of CBD and BRV, all patients were titrated up to at least 17 mg/kg/day of CBD and 40 mg/kg/day of BRV.

Patients were taking an average of four AEDs.

### Clinical Changes

Tables 2A-2C illustrate the seizure frequency for each patient as well as the doses of CBD and BRV given.

TABLE 2A
Seizure frequency data for Patient 1
Patient 1

| | Seizure Type | | |
|---|---|---|---|
| | Focal with secondary | Dose CBD | Dose BRV |
| Time | generalisation | (mg/kg/day) | (mg/kg/day) |
| 108 weeks | 14.4 | 25.0 | 150.0 |
| 120 weeks | 56.0 | 25.0 | 150.0 |
| 132 weeks | 8.0 | 25.0 | 0 |

Patient 1 experienced a 44.4% reduction in focal seizures with secondary generalisation over the 24-week period of concomitant treatment with CBD and BRV.

TABLE 2B
Seizure frequency data for Patient 2
Patient 2

| | Seizure Type | | Dose CBD | Dose BRV |
|---|---|---|---|---|
| Time | Tonic | Tonic-clonic | (mg/kg/day) | (mg/kg/day) |
| 108 weeks | 3.9 | 21.9 | 17.0 | 250.0 |
| 120 weeks | 0 | 13.8 | 15.5 | 200.0 |

Patient 2 experienced a 100% reduction in tonic seizures and a 37.0% reduction in tonic-clonic seizures over the 12-week period of concomitant treatment with CBD and BRV.

TABLE 2C
Seizure frequency data for Patient 3
Patient 3

| | Seizure Type | | | | Dose CBD | Dose BRV |
|---|---|---|---|---|---|---|
| | | | Tonic- | Focal with | (mg/kg/ | (mg/kg/ |
| Time | Atonic | Tonic | clonic | impairment | day) | day) |
| 96 weeks | 70.6 | 85.1 | 13.9 | 113.1 | 40.0 | 40.0 |
| 108 weeks | 28.8 | 30.5 | 8.2 | 277.5 | 38.0 | 20.0 |
| 120 weeks | 63.2 | 549 | 19.4 | 169.0 | 38.0 | 20.0 |
| 132 weeks | 62.2 | 77.3 | 11.8 | 152.9 | 34.5 | 15.0 |
| 144 weeks | 35.2 | 89.5 | 13.4 | 159.4 | 34.5 | 0 |

Patient 3 experienced a 49.7% reduction in atonic seizures over the over the 48-week period of concomitant treatment with CBD and BRV.

Overall, patients reported reductions of 37.0-100.0% in seizures over the period of concomitant treatment with CBD and BRV, during which period the dose of BRV was reduced and the dose of CBD was maintained or reduced.

The treatment was effective in reducing the frequency of the following seizure types: atonic, tonic, tonic-clonic and focal seizures with secondary generalisation. Significantly, one patient became seizure free of tonic seizures (patient 2).

### CONCLUSIONS

These data indicate that CBD combined with a reduced dose of BRV was able to significantly reduce the number of seizures associated with epilepsy. Clearly the treatment is of significant benefit given the high responder rate experienced in all three patients.

In conclusion, this study signifies the use of CBD with a reduced dose of BRV for treatment of seizures associated with epilepsy. Seizure types include atonic, tonic, tonic-clonic, and focal seizures with secondary generalisation for which seizure frequency rates decreased by significant rates, by 37-100.0%.

### REFERENCES

1. Stephen, L. J. & Brodie, M. J. (2018). “Brivaracetam: a novel antiepileptic drug for focal-onset seizures.” Therapeutic Advances in Neurological Disorders, vol. 11; pages 1-10
2. Ryvlin, P., Werhahn, K. J., Blaszczyk, B., Johnson, M. E., Lu, S. (2014) “Adjunctive brivaracetam in adults with uncontrolled focal epilepsy: Results from a double-blind, randomized, placebo-controlled study.” Epilepsia, vol. 55, No. 1, pages 47-56
3. Klotz, K. A., Hirsch, M., Heers, M., Schulze-Bonhage, A., Jacobs, J. (May 2019). Effects of cannabidiol on brivaracetam plasma levels. Epilepsia. 2019; 60:e74-e77.

1. A cannabidiol (CBD) preparation for use in the treatment of seizures associated with epilepsy in patients who are concurrently taking brivaracetam wherein the dose of brivaracetam is lowered.

2. A cannabidiol (CBD) preparation for use according to claim 1, wherein the dose of CBD is lowered.

3. A cannabidiol (CBD) preparation for use according to any of the preceding claims, wherein the seizures associated with epilepsy are atonic, tonic, tonic-clonic seizures and focal seizures with secondary generalisation.

4. A cannabidiol (CBD) preparation for use according to any of the preceding claims, wherein the CBD is in the form of a highly purified extract of cannabis which comprises at least 95% (w/w) CBD.

5. A cannabidiol (CBD) preparation for use according to claim 1, wherein the CBD is present as a synthetic compound.

6. A cannabidiol (CBD) preparation for use according to claim 4, wherein the highly purified extract comprises less than 0.15% tetrahydrocannabinol (THC).

7. A cannabidiol (CBD) preparation for use according to claim 4, wherein the extract further comprises up to 1% cannabidivarin (CBDV).

8. A cannabidiol (CBD) preparation for use according to any of the preceding claims, wherein the CBD is used in combination with one or more concomitant anti-epileptic drugs (AED).

9. A cannabidiol (CBD) preparation for use according to claim 8, wherein the one or more AED is selected from the group consisting of: clobazam, lamotrigine, lacosamide, rufinamide, levetiracetam, diazepam, felbamate and clonazepam.

10. A cannabidiol (CBD) preparation for use according to any of the preceding claims, wherein the dose of CBD is below 50 mg/kg/day.

11. A cannabidiol (CBD) preparation for use according to any of the preceding claims, wherein the dose of CBD is greater than 5 mg/kg/day.

12. A cannabidiol (CBD) preparation for use according to any of the preceding claims, wherein the epilepsy is: Lennox-Gastaut Syndrome; Myoclonic Absence Epilepsy; Tuberous Sclerosis Complex; Dravet Syndrome; Doose Syndrome; Jeavons Syndrome; CDKL5; Dup15q; Neuronal ceroid lipofuscinoses (NCL) and brain abnormalities.

13. A method of treating seizures associated with epilepsy in a patient in need thereof, comprising administering to the patient a therapeutically effective amount of cannabidiol (CBD) with caution, wherein the patient is taking brivaracetam concurrently.

14. The method of claim 13, wherein said caution comprises lowering the dose of CBD.

15. The method of claim 13, wherein said caution comprises lowering the dose of brivaracetam.

16. The method of claim 13, wherein said caution comprises monitoring said patient for side effects.

17. The method of claim 16, wherein said caution further comprises discontinuing CBD if said side effects are observed.

18. The method of claim 13, wherein said caution comprises advising said patient of side effects from said concurrent therapy.
